(19) United States
(12) Patent Application Publication
(10) Pub. No.: US 2023/0255725 A1
(43) Pub. Date: Aug. 17, 2023

(54) DIGITAL TWIN BASED ON ORTHODONTIC ALIGNMENT

(71) Applicant: INTERNATIONAL BUSINESS MACHINES CORPORATION, ARMONK, NY (US)

(72) Inventor: Partho Ghosh, Kolkata (IN)

(21) Appl. No.: 17/650,881

(22) Filed: Feb. 14, 2022

(51) Int. Cl. A61C 7/00; A61C 7/02

(52) U.S. Cl. CPC A61C 7/002; A61C 7/02; A61C 2007/004

(57) ABSTRACT

According to one embodiment, a method, computer system, and computer program product for digital-twin-assisted orthodontic treatment is provided. The present invention may include gathering real-time sensor data on an orthodontic arrangement of a patient; updating an orthodontic digital twin corresponding with the orthodontic arrangement with the sensor data; alerting a user of a complication, compliance with an alignment regimen, or suggested orthodontic procedure based on the orthodontic digital twin; and controlling remote elements to adjust forces acting on the orthodontic arrangement.

### BACKGROUND

The present invention relates, generally, to the field of computing, and more particularly to digital orthodontics.

Orthodontics is a specialty of dentistry concerned with the diagnosis, prevention, and correction of mal-positioned teeth and jaws, and misaligned bite patterns. Orthopedics may also address the modification of facial growth. Digital orthodontics is a field concerned with the combination of digital software with orthodontic hardware to assist in the design, deployment, and operation of dental hardware to ensure correct teeth alignment and prevent tooth irregularity and disproportionate jaw relationships. The combination of certain modern software developments with specialized hardware and traditional orthodontic methods and technology stands to realize significant benefits.

### SUMMARY

According to one embodiment, a method, computer system, and computer program product for digital-twin-assisted orthodontic treatment is provided. The present invention may include gathering real-time sensor data on an orthodontic arrangement of a patient; updating an orthodontic digital twin corresponding with the orthodontic arrangement with the sensor data; alerting a user of a complication, compliance with an alignment regimen, or suggested orthodontic procedure based on the orthodontic digital twin; and controlling remote elements to adjust forces acting on the orthodontic arrangement.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a digital-twin-based orthodontic alignment process according to at least one embodiment;

FIG. 3 is a block diagram of the input components of an orthodontic digital twin according to at least one embodiment;

FIG. 4 is a block diagram of internal and external components of computers and servers depicted in FIG. 1 according to at least one embodiment;

FIG. 5 depicts a cloud computing environment according to an embodiment of the present invention; and

FIG. 6 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to digital orthodontics. The following described exemplary embodiments provide a system, method, and program product to, among other things, create a digital twin of an orthodontic arrangement, run simulations of orthodontic alignment procedures, recommend personalized procedures and compliance regimens for patients, and alert users of issues with ongoing orthodontic procedures. Real-time data of a patient's orthodontic arrangement utilizing sensors within a patient's mouth. The digital twin reflects the status of the physical counterpart by being updated with data in real-time. The digital twin can determine factors affecting the orthodontic arrangement allowing for simulation of the future movement of orthodontic arrangements and guidance for a user to perform suggested orthodontic procedures. Therefore, the present embodiment has the capacity to improve the technical field of digital orthodontics by collecting multiple dimensions of data, including real-time data through sensors of a patient's orthodontic alignment, to build a complete and up-to-date model of a patient's entire orthodontic arrangement and mechanical orthopedic components within the digital twin. This digital twin may be more complete and accurate than other models existing in the field, which may not only make more accurate but may enable entirely simulations

As previously described, orthodontics is a specialty of dentistry concerned with the diagnosis, prevention, and correction of mal-positioned teeth and jaws, and misaligned bite patterns, as well as the modification of facial growth. Digital orthodontics is a field concerned with the combination of digital software with orthodontic hardware to assist in the design, deployment, and operation of dental hardware to ensure correct teeth alignment and prevent tooth irregularity and disproportionate jaw relationships. The combination of certain modern software developments, such as Digital Twin, with specialized hardware and traditional orthodontic methods and technology stands to realize new and significant benefits to the field of orthodontics.

Digital orthodontics has struggled, as a field, to accurately model the dental arrangement. Digital models of a patient's dentition are often incomplete, for example modeling only the teeth, while failing to model the patient's present arrangement of teeth in relation to its supporting bone or alveolar processes, adjacent teeth, and/or opposing dentition. Digital models may also fail to model orthopedic components currently deployed within the patient's orthopedic arrangement, such as brackets, molar bands, arch wire and types, auxiliary arch wire, et cetera. Additionally, digital models may be updated with new information rarely. Data of a patient's orthodontic arrangement is typically only collected at an appointment with an orthopedist or dentist. Since orthodontic appointments are typically separated by weeks or months, this leaves large gaps in patents data. Furthermore, the digital model may be updated incompletely, such that the model may not represent a complete or accurate digital representation of a patient's dentition and/or orthopedic arrangement. It may be difficult or impossible to make accurate predictions and inferences with such incomplete data, and as such, accurate simulation of the patient's dental arrangement over time and/or over the course of a given treatment, as well as accurate estimation of the accuracy and predicted duration of a treatment, may not be feasible. Furthermore, such incomplete data may make timely and accurate detection of complications or diseases, and identification of recommended treatments, difficult or impossible. For example, complications such as loosening or failure of brackets, broken wire, cavities, gum diseases, broken rubber band, et cetera may occur between visits to the orthodontist, and may not become known to the orthodontist until the next appointment, resulting in negative effects ranging from lost progress on an alignment procedure to more severe effects of oral diseases to serious injury from broken equipment.

As such, it may be desirable to implement a system that utilizes digital twin technology to incorporate any of a wide variety of data gathered regarding the entire orthopedic arrangement and any orthopedic components, including data and inferences regarding a patient's demographics, health, daily habits, et cetera, into a digital model comprising a digital twin; the digital twin may be updated in real time or near-real-time by sensors deployed externally or within the orthopedic arrangement such that the digital twin remains an up-to-date, accurate and complete representation of the orthopedic arrangement including orthopedic components. It may further be desirable to implement a system that utilizes the digital twin to simulate the movement of the orthopedic arrangement as a result of forces operating on the orthopedic arrangement over time, the movement of the orthopedic arrangement over the course of a given treatment, the effect of oral diseases or conditions over time, and/or both an accuracy and an estimated duration of any given treatment option. It may further be desirable to implement a system that utilizes the digital twin to automatically modify the orthopedic arrangement through the operation of mechanical orthopedic elements disposed in a patient's mouth, detect and alert patients or users to complications in a timely and accurate fashion, and provide guidance to users and patients to perform orthopedic procedures.

According to one embodiment, the invention may be method of creating a digital twin of a patient's entire orthodontic arrangement; utilizing the digital twin to determine factors affecting the patient's orthodontic arrangement; simulating the future movement of the orthodontic arrangement based on the orthodontic digital twin and the determined factors; and alerting a user of a complication, monitoring compliance with an alignment regimen, and/or recommending an orthodontic procedure based on the simulation, the determined factors, and/or the digital twin.

A digital twin may be a virtual representation of a physical object which is maintained and updated regularly using sensor data, and human observation such that the virtual object is an up-to-date and accurate copy of its physical counterpart; the virtual object can therefore be accessed to ascertain the properties and states of the corresponding physical asset at any given moment in time, making the digital twin concept extremely useful in product design, asset maintenance, asset monitoring, medical diagnostics, digital orthopedics, et cetera. In the context of digital orthodontics, the digital twin may be a virtual representation of a patient's orthodontic arrangement. The digital twin may be created and updated with any input data pertaining to the orthodontic arrangement such as the dimensions, disposition, health, hygiene, ongoing and completed treatment, et cetera of the orthodontic arrangement. This input data may include, for example, patient oral medical data, digital dental imaging, aesthetic profiling, the presence, location, design, composition, and any other related information regarding orthodontic equipment deployed within the orthodontic arrangement such as brackets, molar bands, archwire, auxiliary archwire, retainer, ligatures (elastomeric, steel ties) and auxiliaries (power chain, active coil spring, intermaxillary elastics), bonding agents, et cetera. Input data may further include dental profiling of patients using electronic dental records, electronic dental and medical records, dental scans, tomography and radiograph imaging, causation, dental profiling, pre-existing medical condition, etc. The system may profile digital imaging of the dental scan using optical impressions or cast-based digital impressions, thereby creating a digital copy of the 3D scans that may be more easily mapped to the digital twin.

In some embodiments of the invention, the input data comprising the digital twin may further include impressions of a patient's jaw. The system may be able to collate three-dimensional geometric and quantitative data of the jawbone's condition, as well as the bone mineral density, from electronic dental and medical records. The system may be able to collect and manage an assortment of impressions of a patient's jaw for orthodontic treatment. The system may digitally scan multiple impressions of a jaw, for example a first impression and a second impression, for the orthodontic treatment to compare the two impressions and thereby identify deviations between the first and second impressions that may indicate distortions in the impressions. The system may select the first jaw impression or the second jaw impression as a base impression, replacing distorted tooth data from the base impression with data for the same tooth from a non-base impression.

In some embodiments of the invention, the orthodontic arrangement may comprise a patient's teeth, jaw, and mouth, taking into account teeth positioning and alignment, jaw and bone factors, the relationship between any given tooth and its supporting bone or alveolar process, adjacent teeth, and opposing dentition, and any orthodontic equipment deployed within the patient's mouth and/or jaw. Orthodontic alignment refers to an ideal version of a patient's orthodontic arrangement where the patient's teeth and jaw are positioned to, for example, achieve an even bite, aesthetically pleasing smile, straight teeth, and/or evenly spaced teeth, as well as the process of achieving this ideal orthodontic arrangement. The system may regard a patient's current orthodontic arrangement as an input and work toward orthodontic alignment as the output. Orthodontic alignment may refer to both medically recommended teeth alignment as well as patient-specified cosmetic alignment. Medically recommended teeth alignment may be identified by a user or based on information of past orthodontic alignments stored in the corpus. Cosmetic alignment may be specified by the patient and based on information of past orthodontic alignments stored in the corpus.

In some embodiments of the invention, orthodontic equipment may refer to various functional hardware elements deployed within a patient's orthodontic arrangement, for example bonded to teeth or embedded within the jaw, to facilitate the process of orthodontic alignment. Orthodontic equipment comes in two types: fixed equipment that may be bonded to the teeth or jaws by an orthodontist, and removable equipment that is not bonded to the teeth or jaws and may be removed by the patient. Braces are the most common type of orthodontic equipment, but orthodontic equipment may comprise orthodontic brackets, molar bands, archwire, auxiliary archwire, retainer, ligatures (elastomeric, steel ties), and auxiliaries (power chain, active coil spring, intermaxillary elastics), bonding agent and their specifications.

According to one embodiment, the system may generate a corpus of correlated classifications of various orthodontic digital twins and their corresponding orthodontic plan, materials, accuracy, precautionary habits, time, and virtual alignment progress simulations. The corpus may include data collected related to past digital twins and analysis. The corpus may contain data including historic data related to the orthodontic plan. The orthodontic plan may be a number determined process to achieve orthopedic alignment. The orthodontic plan may also pertain to step by step process to achieve a desired orthodontic alignment and the use of orthodontic equipment. This corpus of data can be then utilized in the analysis of future digital twin applications. Over time the system will gather more data from continuous use of the system that will then affect the analysis and simulation results of future runs of the system. The corpus may collect data on trends in orthodontic alignment plans as well as cosmetic desired trends of patients, patient scheduling trends, and compliance regimen trends.

Sensors may refer to devices, modules, or subsystems capable of detecting and/or measuring events or changes affecting the orthodontic arrangement and transmit the information to the system for addition to the digital twin. The sensors may be deployed within a patient's orthodontic arrangement, for example bonded to the teeth in a dental implant, embedded in the bone of the jaw, affixed to a removable device placed within the mouth such as a retainer, et cetera. The sensors may be capable of measuring forces and movement affecting the orthodontic arrangement, such as wear on the teeth, bite force, jaw action, activity, et cetera. The sensors may measure and/or relay data at regular intervals, which may be close enough together in time to constitute real time or near-real-time updates, for instance on the order of seconds or minutes. The sensors may be equipped with a wireless transmitter to relay the data to the system. The sensors may also identify different types of forces on different parts of the orthodontic arrangement by duration, such as by flagging forces as interrupted, continuous, or intermittent, as well as measuring the magnitude and frequencies of different forces. The sensors may comprise force sensors, motion sensors, cameras, and visual sensors.

According to one embodiment, the system analyzes the data of the digital twin to extract information regarding the patients, oral health, alignment, cosmetic issues, etc. The orthodontic arrangement as represented by the digital twin may be analyzed to determine, for example, the degree of crowding in the patient's teeth and arrangement of oral space; the system may then utilize this information to determine the space required for orthodontic alignment. The system may calculate the course of movement for each tooth and the timeline for each course of movement along with metrics and/or movement materials during any particular stage of the dental alignment process. In some embodiments of the invention, the system may determine an accuracy regarding the timeline and course of movement for each tooth based on variables that affect the dental movement. The system may also analyze real-time forces collected by the sensors, identifying the magnitude and location of the forces within the orthodontic arrangement at different times, and use this information to identify patterns; for example, the system may identify that forces corresponding with biting and chewing occur during discrete time periods at consistent intervals throughout the day; from this, the system may infer the patient's mealtimes and/or snack breaks. In another example, the system may identify a lack of any forces as corresponding with sleep, or severe forces occurring at night to be a sign of bruxism. The system may also analyze personal criteria such as patient's scheduling, personal health issues, and patient-submitted preferences, alone or in conjunction with habits inferred from sensed forces within the orthodontic arrangement, to determine the patient's daily routine to the extent necessary to determine the most convenient times to communicate notifications and alerts to the patients, as well as the most convenient times to operate remote elements of the patient. Personal health issues such as pre-existing conditions, diseases, allergies may be analyzed to determine the most advantageous orthodontic alignment process and minimize the chances of complications and/or the process interacting adversely with any of the patient's existing conditions or health issues. In some embodiments of the invention, the system may identify complications occurring within the orthodontic arrangement based on, at least in part, the sensor data. Complications may be any failure of orthodontic equipment or medical issues arising within the orthodontic arrangement, such as a failure of an orthodontic treatment, health issues such as gum disease, cavities, et cetera. Orthodontic procedures may be procedures related to the positioning of teeth and jaws and bite patterns such as the re-adhesion of braces to a patient's teeth may be an example of an orthodontic procedure, as well as a jawbone density analysis.

Movement materials, as referred to herein, may refer to all mechanisms designed to aid in the movement, rotation, angular correction, et cetera of a tooth. During each stage of the orthodontic alignment, materials such as springs, arch wire, ring/elastic bands et cetera would differ based on their configuration, tensile strength and other properties. The system may be able to suggest different configuration or deployment of the movement materials during different stages of the alignment. For example, during a first stage of an alignment procedure, the spring of diameter 0.3 millimeters with tensile strength of 0.3 newtons of force would be used to push the teeth forward, whereas in a second stage, a lesser force of elastic rubber band can be used, for example a rubber band exerting 0.15 newtons of force.

According to at least one embodiment, the system simulates the future movement of the patient's orthodontic arrangement. This simulation may be based on the current forces acting on the user's orthodontic arrangement and may be used to extrapolate the state of the orthodontic arrangement over time and predict issues arising therefrom, such as tooth crowding, crooked teeth, growing gaps between teeth, et cetera. In some embodiments of the invention, the simulation may model an alignment process. The system may simulate the process of moving the patient's current orthodontic arrangement into a state of orthodontic alignment, and the steps required to transition from the current orthodontic arrangement to orthodontic alignment. The system may, based on the determined steps, generate an orthodontic plan for a user to follow. The simulation may be based on current conditions, which may be based on data from the digital twin and/or factors identified through analysis of the digital twin, and which represent known conditions at the time the simulation is created, such as a type or degree of misalignment, amount of room inside the patient's mouth, degree of overbite or underbite, the distance the patient's teeth must travel to reach orthodontic alignment, dental profiling, age brackets, pre-existing dental conditions like malocclusion, gum diseases, bone conditions, et cetera. In some embodiments of the invention, the system may simulate the effect of dynamic conditions on the alignment process; dynamic conditions may be events that may be unplanned or unexpected that may occur during or before treatment that may affect the course of the treatment, such as wisdom tooth removal before or during movement course, implanted teeth, cavities, gum disease, etc. The proposed system may simulate the procedural steps of orthodontic alignment, its course, and the timeline for each course along with metrics and/or movement materials during that particular stage of dental alignment based on the above.

In some embodiments of the invention, the system may generate an accuracy and proposed timeline for the orthodontic alignment based on the static and dynamic conditions that affect the dental movement, including, for example, the patient's dental hygiene, type of material used in the orthodontic components, the patient's bone growth pattern, and additional demographics of the patient. In some embodiments of the invention, the system may determine the desired space required for orthodontic alignment and thereby recommend pre-orthodontic alignment procedural compliance, such as extraction of particular teeth, timestamped usage of a digital expander, and identification of teeth that need expansion or removal. The orthodontic plan generated by the system may be personalized to account for the user's specific needs or desires. For example, a simulation may be altered if a user specifies only removable equipment to be used. The system may also simulate forces applied to the orthodontic arrangement and the orthodontic alignment, and the forces that affected outcome based on the user's dental profiling, and types of orthodontic procedures. The system would also be able to simulate, monitor, personalize/customize and define appliance configurations or changes to appliance configurations for incrementally moving teeth during the alignment procedure. In some embodiments of the invention, the system may be able to simulate differential lag wherein actual tooth movement lags behind targeted tooth movement at many stages due to patient compliance, treatment procedures, medical conditions etc. In some embodiments of the invention, any simulation may be updated, for example in real time or near real time, and/or whenever the digital twin is updated with new data relevant to the simulation, such that the simulation is based on up-to-date and complete knowledge of the state of the orthodontic arrangement.

According to at least one embodiment, the system may alert a user of complications that may arise during or pertaining to an orthodontic procedure. The proposed system may identify complications occurring presently based on the digital twin, and/or may predict any future complications during or resulting from an orthodontic procedure based on the simulation. Complications may include failure of brackets, loose brackets, broken wires, cavities, gum diseases, broken bands, etc. Responsive to identifying a complication, the system may alert users or patients of the complication by generating an alert on the user or patient's mobile device or computing device. This alert can be audible, textual, or graphical, and may inform the patient or user of the nature, location, associated orthodontic procedure, et cetera. The system may also generate a suggestion for the user or patient to book an appointment for a routine dental visit or emergency visit. The system may also automatically book a routine appointment or emergency for the patient based on the presence and/or severity of the detected complication.

According to one embodiment, the system may identify noncompliance with an alignment regimen, and may alert a patient and/or user of the noncompliance. Compliance regimens may be routines, behaviors, or patterns of behavior suggested to patients to facilitate an orthodontic procedure. For example, a compliance regimen may prescribe a certain diet, or may prescribe the tightening or loosening of braces or the removal of a retainer at certain times or before certain activities such as eating or sleeping or may prescribe brushing teeth or flossing at certain times during the day. The system may generate a number of specific compliance regimens for the patient including but not limited to dietary, supplemental, lifestyle, pre-cautionary changes like avoidable food, specified brushes, avoidance of incisors, chewing patterns, brushing patterns, and amount of time to be on removable braces during different stages of an alignment procedure, etc. The regimens can be suggested based on the digital twin analysis of the patient's orthodontic arrangement, as well as information from the corpus. These regimens can be personalized to specific patient parameters to fit with medical requirements as well as non-medical elective cosmetic requests. The suggested regimens are routines and lifestyle changes that are intended to improve the orthodontic process of the patient. For example, the system may identify that a patient has non-removable braces and may accordingly suggest a regimen to avoid foods that may cause damage to the braces such as chewing gum. The regimens may also be tailored to fit around a patient's personal schedule or lifestyle.

The system may track compliance with the suggested regimen by, for example, querying the user or the patient on patient habits, tracking differential lag between target tooth movement and actual tooth movement during an alignment procedure, status or movement of orthodontic components based on sensor data, forces on the orthodontic arrangement detected by the sensors, et cetera to track elements of the compliance regimen such as diet, amount of time with aligners, bite pressure, hygiene practices of the patient, et cetera. The system may compare this information regarding the patient's compliance habits to the compliance regimen; if the compliance habits do not match the compliance regimen, the system may alert a user and/or patient of the noncompliance and may suggest ways in which the patient may act to remedy the non-compliance and/or to resume compliance with the regimen.

According to at least one embodiment, the system may suggest users perform specific orthodontic procedures to correct or address complications or other health or cosmetic issues identified based on the analysis, simulation, and or/digital twin. These suggested procedures may be based on, for example, real-time data from the system altering the current orthodontic plan. The system may suggest more involved orthodontic procedures, such as installation of implants or bonded braces, be administered by a dental professional, or user; the system may suggest less substantial procedures, such as tightening braces or retainers, to the patient.

According to one embodiment, the system may allow for a user to remotely adjust orthodontic equipment. The system may enable the user to remotely operate different parts of the orthodontic arrangement based on multiple remotely operable devices located on the orthodontic equipment. Remotely operable equipment comprises dental equipment with remotely operable mechanics that can be adjusted. The system may enable the user to manipulate the virtual digital twin and the operations performed on the digital twin will automatically communicate with remotely operable equipment to perform autonomous dental alignment changes with respect to tightening of brackets, springs, screws, or other fastening elements on the braces. The system may remotely operate orthodontic equipment to perform actions including but not limited to tightening, performing angular rotation of brackets, bone growth enhancement such as through an LED bone growth enhancer, pressure reduction, etc. For example, in the event that orthodontic equipment needs to be tightened, the system may instruct the user to remotely tighten the orthodontic equipment, and the user may then remotely adjust the patient's orthodontic equipment. The remote operable orthodontic equipment may communicate, and relay data collected by the system by means of a wireless network. The wireless connection may be through any smart service or wireless data connections between network nodes.

According to one embodiment, the system may be able to remotely guide the user or patient to perform an orthodontic procedure. The system may communicate text, video, and/or audio guides on performing the recommended procedures to the user. The system may also send guides for less intricate procedures that can be self-conducted to the patient. In some embodiments of the invention, the system may remotely guide the user via instructions and graphical elements in a mixed reality environment. Mixed reality may be a field concerned with merging real and virtual worlds such that physical and digital objects co-exist and interact in real time. Mixed reality does not exclusively take place in either the physical or virtual worlds but is a hybrid of reality and virtual reality; as such, mixed reality describes everything in the reality-virtuality continuum except for the two extremes, namely purely physical environments and purely virtual environments.

The system may utilize mixed reality to place instructions in the field of vision of the user or patient, and/or overlay graphical elements onto the user or patient's vision for any number of steps comprising the orthopedic procedure to indicate orthopedic components or elements of the orthopedic arrangement such as teeth or jaws that the user or patient are to interact with, illustrate actions or motions to be taken in completing the step, warnings and advice, et cetera. For example, the system may guide users through an orthodontic process for correcting orthodontic equipment which needs frequent removal, reapplication, and/or replacement. The system may also guide the user to change orthodontic equipment like bands, springs, retainers before and/or after events such as meals, brushing, flossing, et cetera. The mixed reality environment, and/or any graphical or textual overlays, may be generated by a mixed reality device.

A mixed-reality device may be a device comprising a display visible to the user or patient that can render a virtual environment and/or overlay virtual elements onto a physical environment visible to the user. The mixed-reality device may further be capable of tracking the location and motion of itself relative to the physical world, and by extension relative to virtual objects mapped to locations in the physical world. The mixed reality device may be a general-purpose device owned by the user, such as a cell phone, or may be customized or specialized for an individual mixed reality experience or class of mixed reality experiences. Mixed reality devices may include such devices as VR headsets, AR headsets, smart glasses, tablets, mobile phones, et cetera.

According to at least one embodiment of the invention, the orthopedic components may include fixed orthodontic braces. Fixed orthodontic braces may be devices used in orthodontics that align and straighten teeth and comprising of brackets attached to the outside surfaces of the teeth or they can be the backside of teeth and maybe made of stainless steel, ceramic (clear or tooth-colored) or other materials, the bracket consisting of a hook for the attachment of auxiliaries such as coil springs and elastics, identification marks that may be colored dots or indentations placed to ensures that the brackets are placed in the correct orientation on the tooth, slots where the orthodontic archwire is placed, tie wings to secure the wire into the bracket slot, bracket adhesion applied to teeth using glue, ring-like bands that encircle the molar teeth, springs placed on the archwire of the braces that may apply pressure between two teeth, pressing the teeth apart and adding space, flexible wire (archwire) that connects all of the brackets and bands, small rubber bands (elastic ties) or metal ties to secure the wire to the brackets (though some braces have a sliding mechanism instead of ties to secure the wire, headgear attached to the braces attached to headgear tubes in the mouth to produce additional pressure and temporary anchorage devices (TADs) being tiny screws are placed through the gums into the jawbone, and can then be used as anchors to apply continuous pressure to move the teeth. Braces may be adjusted periodically by tightening or bending the interconnecting wires. This puts mild pressure on the teeth and gradually shifts them into new positions. The jaw responds to the pressure by dissolving bone in the path of the moving tooth and laying down new bone behind it. Users may use the tension between the upper and lower jaws to help correct alignment with elastic bands stretched between opposing teeth. According to one embodiment, the orthopedic components may include retainers. Retainers may be a removable, custom-made appliances typically made of plastic or plastic and metal wires that help teeth stabilize for a period of time to prevent them from shifting back to their original position.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product to create a digital twin of an orthodontic arrangement, run simulations of orthodontic alignment procedures, recommend personalized procedures and compliance regimens for patients, and alert users of issues with ongoing orthodontic procedures.

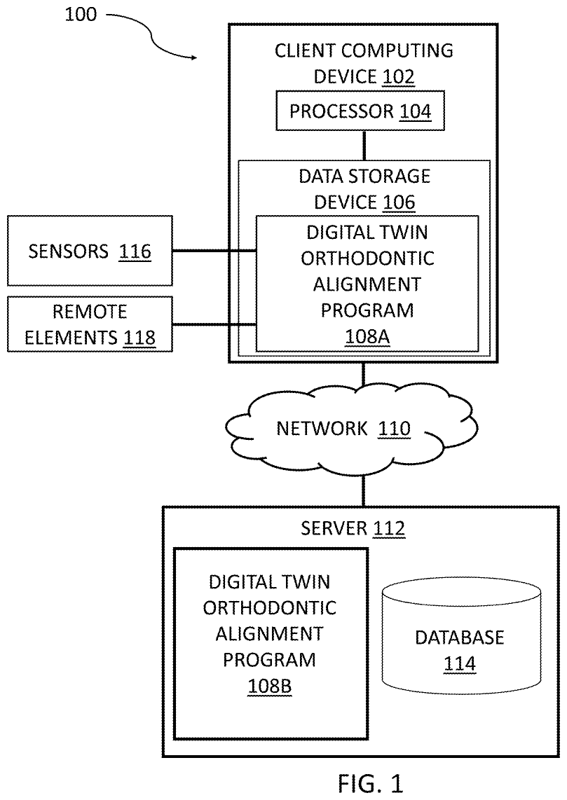

Referring to FIG. 1, an exemplary networked computer environment 100 is depicted, according to at least one embodiment. The networked computer environment 100 may include client computing device 102, a server 112, sensors 116, and remote elements 118, interconnected via a communication network 110. According to at least one implementation, the networked computer environment 100 may include a plurality of client computing devices 102, servers 112, sensors 116, and remote elements 118, of which only one of each is shown for illustrative brevity.

The communication network 110 may include various types of communication networks, such as a wide area network (WAN), local area network (LAN), a telecommunication network, a wireless network, a public switched network and/or a satellite network. The communication network 110 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

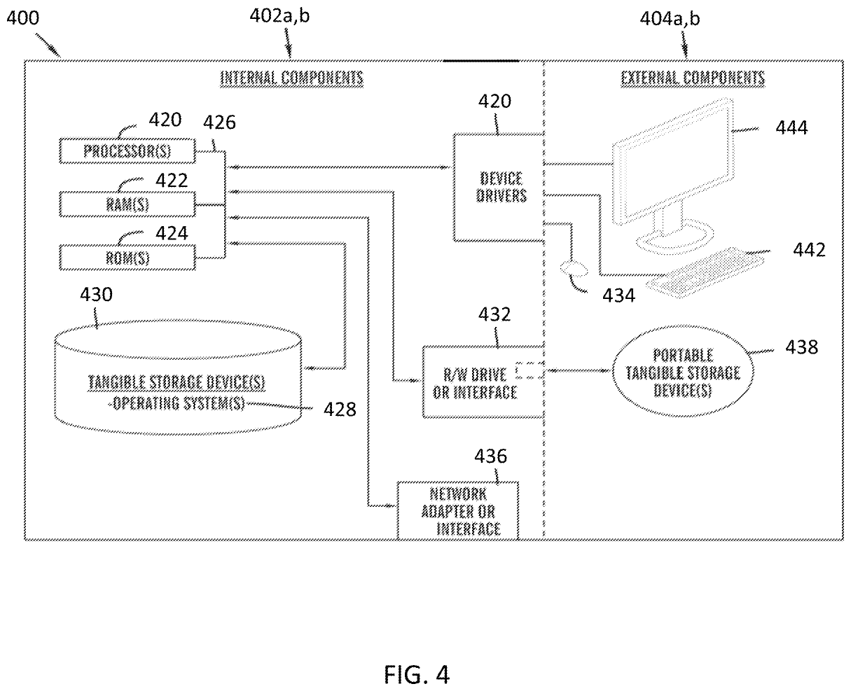

Client computing device 102 may include a processor 104 and a data storage device 106 that is enabled to host and run a digital-twin-based orthopedic alignment program 108A and communicate with the server 112 via the communication network 110, in accordance with one embodiment of the invention. Client computing device 102 may be, for example, a mobile device, a telephone, a personal digital assistant, a netbook, a laptop computer, a tablet computer, a desktop computer, or any type of computing device capable of running a program and accessing a network. As will be discussed with reference to FIG. 4, the client computing device 102 may include internal components 402a and external components 404a, respectively.

The server computer 112 may be a laptop computer, netbook computer, personal computer (PC), a desktop computer, or any programmable electronic device or any network of programmable electronic devices capable of hosting and running a digital-twin-based orthopedic alignment program 108B and a database 116 and communicating with the client computing device 102 via the communication network 110, in accordance with embodiments of the invention. As will be discussed with reference to FIG. 4, the server computer 112 may include internal components 402b and external components 404b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

Sensors 116 may refer to devices, modules, or subsystems capable of detecting and/or measuring events or changes affecting the orthodontic arrangement and transmit the information to the digital-twin-based orthopedic alignment program 108A, 108B for addition to the digital twin. The sensors 116 may be deployed within a patient's orthodontic arrangement, for example bonded to the teeth in a dental implant, embedded in the bone of the jaw, affixed to a removable device placed within the mouth such as a retainer, et cetera. The sensors 116 may be capable of measuring forces and movement affecting the orthodontic arrangement, such as wear on the teeth, bite force, jaw action, activity, et cetera. The sensors 116 may measure and/or relay data at regular intervals, which may be close enough together in time to constitute real time or near-real-time updates, for instance on the order of seconds or minutes. The sensors 116 may be equipped with a wireless transmitter to relay the data to the digital-twin-based orthopedic alignment program 108A, 108B, for example over network 110. The sensors 116 may also identify different types of forces on different parts of the orthodontic arrangement by duration, such as by flagging forces as interrupted, continuous, or intermittent, as well as measuring the magnitude and frequencies of different forces. The sensors 116 may comprise force sensors, motion sensors, cameras, and visual sensors.

The remote elements 118 may be mechanical components fitted to a user's teeth or orthodontic alignment which, when operated, can move or reposition to produce changes in the user's orthodontic arrangement. The remote elements 118 may be equipped with transmitters and may be connected to the network 110 such that they may communicate with and be remotely controlled by digital-twin-based orthopedic alignment program 108A, 108B. In some embodiments, the digital-twin-based orthopedic alignment program 108A, 108B may enable the user to manipulate the virtual digital twin and the operations performed on the digital twin will auto communicate with the remote elements 118 to produce autonomous dental alignment changes, for example with respect to tightening of brackets, springs, screws, or other fastening elements on the braces. The digital-twin-based orthopedic alignment program 108A, 108B may remotely operate the remote elements 118 to perform actions including but not limited to tightening, performing angular rotation of brackets, bone growth enhancement such as through an LED bone growth enhancer, pressure reduction, etc. For example, in the event that orthodontic equipment needs to be tightened, the digital-twin-based orthopedic alignment program 108A, 108B may notify the user remotely to tighten the orthodontic equipment, and the user may then remotely adjust the patient's orthodontic equipment via remote elements 118.

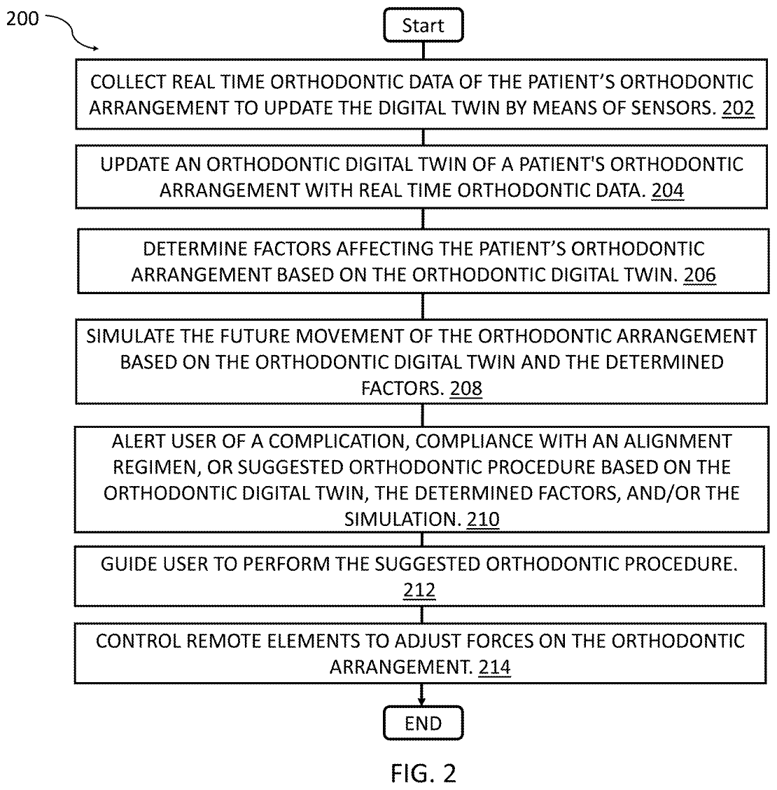

According to the present embodiment, the digital-twin-based orthopedic alignment program 108A, 108B may be a program capable of create a digital twin of an orthodontic arrangement, run simulations of orthodontic alignment procedures, recommend personalized procedures and compliance regimens for patients, and alert users of issues with ongoing orthodontic procedures. The digital-twin-based orthopedic alignment program 108A, 108B may be located on client computing device 102 or server 112 or on any other device located within network 110. Furthermore, digital-twin-based orthopedic alignment program 108A, 108B may be distributed in its operation over multiple devices, such as client computing device 102 and server 112. The digital-twin-based orthodontic alignment method is explained in further detail below with respect to FIG. 2.

Referring now to FIG. 2, an operational flowchart illustrating a digital-twin-based orthodontic alignment process 200 is depicted according to at least one embodiment. At 202, the digital-twin-based orthopedic alignment program 108A, 108B collects real-time orthodontic data of the patient's orthodontic arrangement to update the digital twin by means of sensors 116. Here, the digital-twin-based orthopedic alignment program 108A, 108B may utilize sensors 116 to dynamically monitor the magnitude and duration of forces acting on the patient's orthodontic arrangement. Dynamic monitoring may include taking sensor readings at regular and frequent time intervals, for example every second or every 5 seconds.

At 204, digital-twin-based orthopedic alignment program 108A, 108B updates an orthodontic digital twin of a patient's orthodontic arrangement with real time orthodontic data. The digital-twin-based orthopedic alignment program 108A, 108B may update the orthodontic digital twin with collected sensor readings as they are received from sensors 116 such that the orthodontic digital twin is current to within a margin of a few seconds.

At 206, digital-twin-based orthopedic alignment program 108A, 108B determines factors affecting the patient's orthodontic arrangement based on the orthodontic digital twin. Here, the digital-twin-based orthopedic alignment program 108A, 108B may analyze the data of the orthodontic digital twin to extract factors regarding the patient's oral health, alignment, cosmetic issues, etc. These factors may include the type or degree of misalignment, amount of room inside the patient's mouth, a degree of over or underbite, a distance the patient's teeth must travel, dental profiling, age brackets, pre-existing dental conditions like malocclusion, gum diseases, bone conditions et cetera, and dynamic conditions like wisdom tooth before or during movement course, implanted teeth, et cetera. Factors may include personal criteria such as a patient's scheduling, personal health issues, complications, and tracking compliance regimens

At 208, digital-twin-based orthopedic alignment program 108A, 108B simulates the future movement of the orthodontic arrangement based on the orthodontic digital twin and the determined factors. The digital-twin-based orthopedic alignment program 108A, 108B may calculate the course of movement for each tooth and the timeline for each course of movement along with metrics and/or movement materials during any particular stage of the dental alignment process. In some embodiments of the invention, the system may determine an accuracy regarding the timeline and/or the course of movement for each tooth based on variables that affect the dental movement.

At 210, digital-twin-based orthopedic alignment program 108A, 108B alerts users of a detected or predicted complication, compliance with an alignment regimen, or suggested orthodontic procedure based on the orthodontic digital twin, the determined factors, and/or the simulation. Based on factors such as personal health issues such as pre-existing conditions, diseases, allergies, cavities, eating habits, the current or projected alignment of the patient's teeth, et cetera, the digital-twin-based orthopedic alignment program 108A, 108B may identify a need for a particular orthodontic procedure. In embodiments where the patient is already participating in an ongoing procedure such as an alignment regimen, the digital-twin-based orthopedic alignment program 108A, 108B may monitor compliance with the regimen and notify the user if the patient is not complying with the ongoing procedure, and/or if complications have arisen in the procedure; based on the factors and/or the simulation, the digital-twin-based orthopedic alignment program 108A, 108B may identify complications in the alignment regime or in a potential orthodontic procedure, which may be a failure of orthodontic equipment or an unfavorable medical result arising from the ongoing procedure. Ongoing procedures may include procedures related to the positioning of teeth and jaws and bite patterns such as the re-adhesion of braces to a patient's teeth, teeth alignment, jawbone density analysis, et cetera. The digital-twin-based orthopedic alignment program 108A, 108B may notify the user via text, graphics, and/or audio sent to the user's mobile device or to any computing device capable of interfacing with the user. In some embodiments, the digital-twin-based orthopedic alignment program 108A, 108B may alert a patient of a detected or predicted complication, compliance with an alignment regimen, or suggested orthodontic procedure based on the orthodontic digital twin, the determined factors, and/or the simulation; in such embodiments, the digital-twin-based orthopedic alignment program 108A, 108B analyze a patient's scheduling to determine the most advantageous times to notify the patient.

At 212, digital-twin-based orthopedic alignment program 108A, 108B guides the user to perform the suggested orthodontic procedure. Here, digital-twin-based orthopedic alignment program 108A, 108B may provide textual, graphical, and/or audio instructions, and/or a combination of all three, to a user to perform the suggested orthodontic procedure on the patient. In some embodiments of the invention, for example where a procedure is simple and safe and can be performed manually, the digital-twin-based orthopedic alignment program 108A, 108B may guide the user to perform the procedure on herself. In some embodiments of the invention, the digital-twin-based orthopedic alignment program 108A, 108B may provide graphical, audio, and/or text elements overlaid onto or otherwise incorporated into a mixed reality environment to guide the user to perform the suggested orthodontic procedure.

At 214, digital-twin-based orthopedic alignment program 108A, 108B controls remote elements 118 to adjust forces on the orthodontic arrangement. Here, digital-twin-based orthopedic alignment program 108A, 108B may remotely instruct remote elements 118 to adjust forces acting on the patient's orthodontic arrangement in accordance with the suggested orthodontic procedure, the alignment regimen, et cetera. In some embodiments of the invention, the digital-twin-based orthopedic alignment program 108A, 108B may analyze the real-time forces acting on the patient's orthodontic arrangement to extrapolate a schedule, and/or may extract information from a patient's online schedule or from patient prompts to determine an opportunity to control the remote elements 118 which would cause minimal disruption for the patient, for example a time when the patient is sleeping, a time between meals, a time when the patient is not at work or otherwise busy, et cetera.

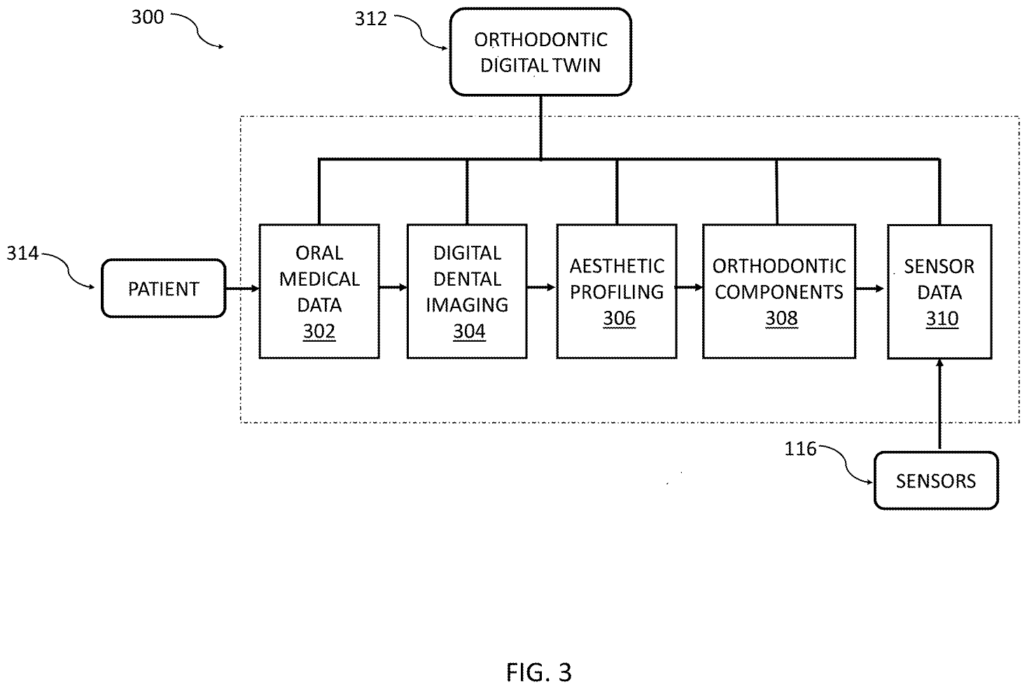

Referring now to FIG. 3, a block diagram 300 of the input components of an orthodontic digital twin 312 is depicted according to at least one embodiment. The orthodontic digital twin 312 is comprised of information from a variety of sources regarding the orthodontic arrangement of a patient 314, including oral medical data 302, digital dental imaging 304, aesthetic profiling 306, orthodontic components 308, and sensor data 310, which is gathered by sensors 116.

It may be appreciated that FIGS. 2-3 provide only illustrations of individual implementations and do not imply any limitations with regard to how different embodiments may be implemented. Many modifications to the depicted environments may be made based on design and implementation requirements.

FIG. 4 is a block diagram 400 of internal and external components of the client computing device 102 and the server 112 depicted in FIG. 1 in accordance with an embodiment of the present invention. It should be appreciated that FIG. 4 provides only an illustration of one implementation and does not imply any limitations with regard to the environments in which different embodiments may be implemented. Many modifications to the depicted environments may be made based on design and implementation requirements.

The data processing system 402, 404 is representative of any electronic device capable of executing machine-readable program instructions. The data processing system 402, 404 may be representative of a smart phone, a computer system, PDA, or other electronic devices. Examples of computing systems, environments, and/or configurations that may represented by the data processing system 402, 404 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 402 a,b and external components 404 a,b illustrated in FIG. 4. Each of the sets of internal components 402 include one or more processors 420, one or more computer-readable RAMs 422, and one or more computer-readable ROMs 424 on one or more buses 426, and one or more operating systems 428 and one or more computer-readable tangible storage devices 430. The one or more operating systems 428 and the digital-twin-based orthopedic alignment program 108A in the client computing device 102, and the digital-twin-based orthopedic alignment program 108B in the server 112 are stored on one or more of the respective computer-readable tangible storage devices 430 for execution by one or more of the respective processors 420 via one or more of the respective RAMs 422 (which typically include cache memory). In the embodiment illustrated in FIG. 4, each of the computer-readable tangible storage devices 430 is a magnetic disk storage device of an internal hard drive. Alternatively, each of the computer-readable tangible storage devices 430 is a semiconductor storage device such as ROM 424, EPROM, flash memory or any other computer-readable tangible storage device that can store a computer program and digital information.

Each set of internal components 402 a,b also includes a R/W drive or interface 432 to read from and write to one or more portable computer-readable tangible storage devices 438 such as a CD-ROM, DVD, memory stick, magnetic tape, magnetic disk, optical disk or semiconductor storage device. A software program, such as the digital-twin-based orthopedic alignment program 108A, 108B, can be stored on one or more of the respective portable computer-readable tangible storage devices 438, read via the respective R/W drive or interface 432, and loaded into the respective hard drive 430.

Each set of internal components 402 a,b also includes network adapters or interfaces 436 such as a TCP/IP adapter cards, wireless Wi-Fi interface cards, or 3G or 4G wireless interface cards or other wired or wireless communication links. The digital-twin-based orthopedic alignment program 108A in the client computing device 102 and the digital-twin-based orthopedic alignment program 108B in the server 112 can be downloaded to the client computing device 102 and the server 112 from an external computer via a network (for example, the Internet, a local area network or other, wide area network) and respective network adapters or interfaces 436. From the network adapters or interfaces 436, the digital-twin-based orthopedic alignment program 108A in the client computing device 102 and the digital-twin-based orthopedic alignment program 108B in the server 112 are loaded into the respective hard drive 430. The network may comprise copper wires, optical fibers, wireless transmission, routers, firewalls, switches, gateway computers and/or edge servers.

Each of the sets of external components 404 a,b can include a computer display monitor 444, a keyboard 442, and a computer mouse 434. External components 404 a,b can also include touch screens, virtual keyboards, touch pads, pointing devices, and other human interface devices. Each of the sets of internal components 402 a,b also includes device drivers 440 to interface to computer display monitor 444, keyboard 442, and computer mouse 434. The device drivers 440, R/W drive or interface 432, and network adapter or interface 436 comprise hardware and software (stored in storage device 430 and/or ROM 424).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

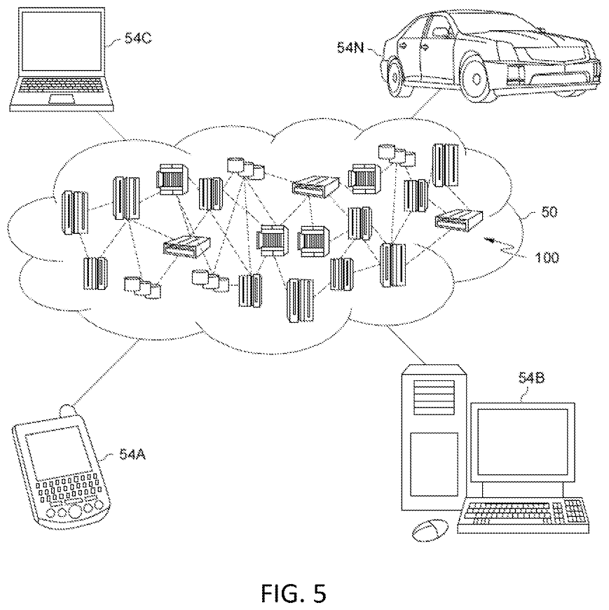

Referring now to FIG. 5, illustrative cloud computing environment 50 is depicted. As shown, cloud computing environment 50 comprises one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 100 and cloud computing environment 50 can communicate with any type of computerized device over any type of network and/or network addressable connection (e.g., using a web browser).

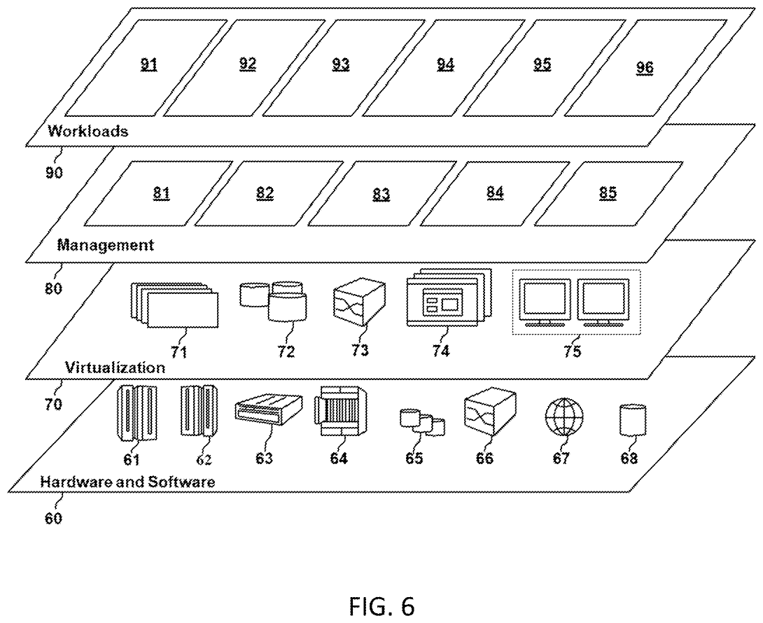

Referring now to FIG. 6, a set of functional abstraction layers 600 provided by cloud computing environment 50 is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and digital-twin-based orthodontic alignment 96. The digital-twin-based orthopedic alignment 96 may be enabled to create a digital twin of an orthodontic arrangement, run simulations of orthodontic alignment procedures, recommend personalized procedures and compliance regimens for patients, and alert users of issues with ongoing orthodontic procedures.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for digital-twin-assisted orthodontic treatment, the method comprising:
gathering real-time sensor data on an orthodontic arrangement of a patient;
updating an orthodontic digital twin corresponding with the orthodontic arrangement with the sensor data;
alert a user of a complication, compliance with an alignment regimen, or suggested orthodontic procedure based on the orthodontic digital twin; and
controlling remote elements to adjust forces acting on the orthodontic arrangement.

2. The method of claim 1, further comprising:
simulating the future movement of the orthodontic arrangement based on the orthodontic digital twin.

3. The method of claim 2, further comprising:
calculating an accuracy of the simulation regarding the timeline or course of movement for each tooth of the orthodontic arrangement.

4. The method of claim 1, wherein the complication or compliance is predicted based on the simulation.

5. The method of claim 1, wherein the orthodontic digital twin comprises real-time sensor data, oral medical data, digital dental imaging, aesthetic profiling, and information regarding orthodontic components.

6. The method of claim 1, further comprising:
based on the digital twin and the patient's schedule, determining a time of day to control the remote elements to decrease patient discomfort.

7. The method of claim 1, further comprising:
guiding the user to perform the suggested orthodontic procedure utilizing mixed reality elements.

8. A computer system for digital-twin-assisted orthodontic treatment, the computer system comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
gathering real-time sensor data on an orthodontic arrangement of a patient;
updating an orthodontic digital twin corresponding with the orthodontic arrangement with the sensor data;
alert a user of a complication, compliance with an alignment regimen, or suggested orthodontic procedure based on the orthodontic digital twin; and
controlling remote elements to adjust forces acting on the orthodontic arrangement.

9. The computer system of claim 8, further comprising:
simulating the future movement of the orthodontic arrangement based on the orthodontic digital twin.

10. The computer system of claim 8, further comprising:
calculating an accuracy of the simulation regarding the timeline or course of movement for each tooth of the orthodontic arrangement.

11. The computer system of claim 8, wherein the complication or compliance is predicted based on the simulation.

12. The computer system of claim 8, wherein the orthodontic digital twin comprises real-time sensor data, oral medical data, digital dental imaging, aesthetic profiling, and information regarding orthodontic components.

13. The computer system of claim 8, further comprising:
based on the digital twin and the patient's schedule, determining a time of day to control the remote elements to decrease patient discomfort.

14. The computer system of claim 8, further comprising:
guiding the user to perform the suggested orthodontic procedure utilizing mixed reality elements.

15. A computer program product for digital-twin-assisted orthodontic treatment, the computer program product comprising:
one or more computer-readable tangible storage medium and program instructions stored on at least one of the one or more tangible storage medium, the program instructions executable by a processor to cause the processor to perform a method comprising:
gathering real-time sensor data on an orthodontic arrangement of a patient;
updating an orthodontic digital twin corresponding with the orthodontic arrangement with the sensor data;
alert a user of a complication, compliance with an alignment regimen, or suggested orthodontic procedure based on the orthodontic digital twin; and
controlling remote elements to adjust forces acting on the orthodontic arrangement.

16. The computer program product of claim 15, further comprising: simulating the future movement of the orthodontic arrangement based on the orthodontic digital twin.

17. The computer program product of claim 15, further comprising:
calculating an accuracy of the simulation regarding the timeline or course of movement for each tooth of the orthodontic arrangement.

18. The computer program product of claim 15, wherein the complication or compliance is predicted based on the simulation.

19. The computer program product of claim 15, wherein the orthodontic digital twin comprises real-time sensor data, oral medical data, digital dental imaging, aesthetic profiling, and information regarding orthodontic components.

20. The computer program product of claim 15, further comprising:
based on the digital twin and the patient's schedule, determining a time of day to control the remote elements to decrease patient discomfort.
